(19) United States
(12) Patent Application Publication
(10) Pub. No.: US 2010/0249252 A1
(43) Pub. Date: Sep. 30, 2010

(54) ZR-FE CATALYSTS FOR FISCHER-TROPSCH SYNTHESIS

(76) Inventors: James G. Goodwin, JR., Clemson, SC (US); Edgar Lotero, Cleveland, OK (US); Nattaporn Lohitharn, Ashburn, VA (US)

Correspondence Address: DORITY & MANNING, P.A., POST OFFICE BOX 1449, GREENVILLE, SC, 29602-1449, US

(21) Appl. No.: 12/599,513

(22) PCT Filed: Jun. 12, 2008
(86) PCT No.: PCT/US08/66659

§ 371 Date: Nov. 10, 2009

Related U.S. Application Data
(60) Provisional application No. 60/934,265, filed on Jun. 12, 2007.

(51) Int. Cl. C07C 1/04; B01J 23/34; B01J 23/745; B01J 21/08

(52) U.S. Cl. 518/721; 502/338; 502/331; 502/324; 502/316; 502/330; 502/337; 502/242

(57) ABSTRACT

Disclosed are solid titanium-free Fischer-Tropsch catalysts including iron homogeneously modified with a zirconium promoter/stabilizer. The homogeneously mixed solid catalysts can be formed through co-precipitation of iron and zirconium precursors followed by calcination and reduction to form the active catalyst materials. The catalysts can optionally include additional materials such as copper, potassium, and silicon promoters.

### CROSS REFERENCE TO RELATED APPLICATION

This application claims filing benefit of U.S. Provisional Patent Application Ser. No. 60/934,265 having a filing date of Jun. 12, 2007.

### FEDERALLY SPONSORED RESEARCH AND DEVELOPMENT

The United States Government may have rights in this disclosure pursuant to grants provided by the National Association of State Energy Offices (grant no. DE-FC36-03G013026).

### BACKGROUND

Petrochemicals, formed from petroleum raw materials, play a major role in the modern world. Formation processes based on alternative raw materials are currently being sought that can provide the desired products without relying on an increasingly unstable petroleum feedstock. One of the most promising routes for maintaining hydrocarbon production while simultaneously decreasing dependence on depleting petroleum reserves is through catalytic conversion of synthesis gas (syngas) to form liquid fuels as well as other hydrocarbons.

Syngas, a CO-rich CO/H₂ mixture, is typically converted to hydrocarbons according to Fischer-Tropsch synthesis (FTS) processes. The selective hydrogenation of CO afforded by FTS has been extensively studied. Generally, bulk iron catalysts have been the catalysts of choice for the conversion of low H₂/CO ratio syngas due to the high water-gas shift (WGS) activity, high FTS activity, and relatively low costs, for instance as compared to cobalt catalysts.

Addition of promoters and/or stabilizers to FTS catalysts has shown promise for increased application of FTS processes on an industrial scale. For instance, the addition of metal promoters such as manganese to iron catalysts is believed to improve the selectivity of light alkenes in the conversion process. The addition of alkali metals, particularly potassium, and/or copper to iron catalysts is also believed to lead to increased FTS reaction rates. (See, e.g., Wang, et al. Catalysis Letters, Vol. 105, Nos. 1-2, November 2005.) Other catalysts have also been developed. For instance, U.S. Pat. No. 4,624,942 to Dyer, et al. discloses a Fischer-Tropsch catalyst comprising iron co-deposited with or deposited on particles comprising a mixture of zirconia and titanic.

While improvements have been made in various aspects of Fischer-Tropsch synthesis processes, room for improvement in the field still exists. What are needed in the art are iron-based catalysts having high FTS activity, low methane selectivity, and long-term stability.

### SUMMARY

Disclosed herein are Fischer-Tropsch synthesis (FTS) catalysts, methods for forming the FTS catalysts, and methods for using the catalysts. For instance, in one embodiment, disclosed is an FTS catalyst that includes zirconium inserted throughout an iron oxide bulk catalyst, the catalyst including zirconium in an amount between about 3 mol % and about 20 mol % of the total amount of iron and zirconium in the catalyst. In addition, catalysts disclosed herein are titanium-free catalysts.

Also disclosed are methods for forming the catalyst. For instance, a method can include forming or providing a mixture including an iron precursor and a zirconium precursor in solution. For example, a mixture can include an iron salt and a zirconium salt in solution. Upon addition of a suitable precipitation initiator, the iron and zirconium can co-precipitate. The precipitated product can then be heat treated to convert the metals to metal oxides, followed by activation to form the FTS catalysts.

Disclosed catalysts can be used in any FTS process. For instance, disclosed catalysts can be used in a two-phase process, in which the product hydrocarbons are all gaseous, or alternatively in a three phase process, in which one or more of the hydrocarbon products are in a liquid phase.

### BRIEF DESCRIPTION OF THE FIGURES

A full and enabling disclosure of the present subject matter, including the best mode thereof, to one of ordinary skill in the art, is set forth more particularly in the remainder of the specification, including reference to the accompanying figures, in which:

FIG. 1 illustrates the total hydrocarbon (C₁-C₈) formation rates for FTS processes using various catalysts;

FIG. 2 illustrates carbon dioxide formation rates for WGS reactions during FTS processes using various catalysts;

FIG. 3 compares the total hydrocarbon (C₁-C₈) formation rates for FTS processes using various catalysts with and without potassium added to the catalysts;

FIG. 4 compares carbon dioxide formation rates for WGS reactions during FTS processes using various catalysts with and without potassium added to the catalysts;

FIG. 5 compares the impact of potassium addition to Mn- and Zr-promoted iron catalysts on the formation rate of total hydrocarbons in an FTS process; and

FIG. 6 compares the impact of potassium addition to Mn- and Zr-promoted iron catalysts on the formation rate of carbon dioxide in an FTS process.

### DETAILED DESCRIPTION

Reference will now be made in detail to various embodiments of the disclosed subject matter, one or more examples of which are set forth below. Each embodiment is provided by way of explanation, not limitation, of the subject matter. In fact, it will be apparent to those skilled in the art that various modifications and variations may be made without departing from the scope or spirit of the disclosure. For instance, features illustrated or described as part of one embodiment, may be used in another embodiment to yield a still further embodiment.

The present disclosure is generally directed to Fischer-Tropsch catalysts that can show improved reactivity as compared to previously known catalysts. More specifically, the disclosed catalysts are titanium free, solid iron catalysts that have been modified with zirconium such that there can be a homogeneously mixed concentration of zirconium throughout the bulk catalyst material.

While not wishing to be bound by any particular theory, it is believed that due at least in part to the regular distribution of zirconium and iron throughout the disclosed catalysts, and the lack of titanium in the catalysts, the Fe/Zr catalysts can produce a higher FTS activity as well as increased stability as compared to other, previously known iron catalysts. Formation processes suitable for the disclosed catalysts can include those capable of providing zirconium inserted throughout the iron oxide bulk catalyst. The addition of zirconium to catalysts as herein described can not only improve overall reactivity of the catalysts, but can also have a positive impact on chain growth probability of the product hydrocarbons as well as reduce induction period behavior of the catalysts as compared to other iron catalysts.

According to one embodiment, disclosed Zr/Fe catalysts can be formed according to a co-precipitation formation process. In particular, zirconium and iron precursors can be co-precipitated from a solution including a mixture of the dissolved starting materials. Suitable starting materials can be those that can be dissolved in solution for subsequent co-precipitation according to the formation process. In addition, the starting materials can be provided to the solution in an amount so as to provide a precipitated product including zirconium in a concentration of between about 3 mol % and about 20 mol % by the total molar amount of iron and zirconium, or in a concentration of between about 2.7 wt. % and about 18.2 wt % by the total weight of iron and zirconium.

For example, in one embodiment, the formed titanium free catalyst can be a bimetallic catalyst having the composition:
(100−x)Fe/xZr wherein x is between about 3 mol % and about 20 mol %
   Or alternatively having the composition:
(100−y)Fe/yZr wherein y is between about 2.7 wt. % and about 18.2 wt %

Accordingly, the iron and zirconium-containing starting materials can provide the two metals in an appropriate ratio to one another upon co-precipitation of a homogeneously mixed solid precursor from solution.

Suitable starting materials/precursors can include, for example, iron and zirconium salts that can be co-precipitated from solution by addition of suitable precipitation initiator. In one preferred embodiment, the precipitation initiator can include ammonium hydroxide, for instance in those embodiments in which the co-precipitation of precursors is pH initiated. The metal precursors can include, without limitation metal nitrates, oxalates, sulphates, chlorides, alkoxides, acetates, benzoates, maleates, and the like. In addition, it should be understood that iron precursors can provide iron in a ferrous form, a ferric form, or a combination thereof.

A co-precipitation process can be carried out at any temperature. In one preferred embodiment, however, a co-precipitation process can be carried out at an elevated temperature, for instance between about 50° C. and about 100° C., or between about 70° C. and about 90° C., in another embodiment.

In one embodiment, additional materials can be included in the catalysts disclosed herein. For instance, other metals and/or metalloids such as silicon, copper, manganese, chromium, cobalt, nickel, vanadium, tantalum, potassium, sodium, cesium, and/or molybdenum can be incorporated in the catalysts. When included, additional metals and metalloids can be incorporated into the catalysts in amounts as are generally known in the art. For instance, copper can be incorporated into a catalyst in an amount of about 3 to about 7 mol % or about 2 to about 5 wt % by weight of the catalyst, typically at about 5 mol % or about 3 wt % by weight of the catalyst, silicon can be incorporated into a catalysts in an amount of about 10 to about 35 mol % or about 3 to about 10 wt % by weight of the catalyst, more commonly at about 17 mol % or about 5 wt % by weight of the catalyst, and potassium can be incorporated into the catalyst in an amount of about 1 to about 10 mol % or about 0.4-4 wt %, typically about 4 mol % or about 2 wt % by weight of the catalyst.

Additional material can be incorporated into the catalysts according to any suitable method. Preferred methods for incorporation of the materials can generally depend upon the particular materials to be included. For instance, certain metals and metalloids such as copper, silicon, and the like can be co-precipitated with the zirconium and iron precursors and can be homogeneously incorporated throughout the bulk catalysts. Optionally, additional materials can be deposited or impregnated on the catalysts following co-precipitation. For example, co-precipitated Fe/Zr precursor can be impregnated with an alkali metal salt such as KHCO₃ according to methods as are generally known to those of skill in the art. In those embodiments in which an additional material is deposited on or impregnated into the co-precipitation product, it may be beneficial to dry the precipitated products first, for instance in an oven held at a temperature of between about 90° C. and about 110° C.

The surface area of an un-promoted Fe catalyst (e.g., 100Fe/5Cu/17Si) is typically about 320 m²/g. When incorporating a Zr promoter, as described herein, the surface area can increase, for instance to about 350 m²/g. Particle size of the formed materials can generally be less than about 150 μm in size, for instance between about 50 μm and about 100 μm in size. In one embodiment, formed materials can be treated, e.g., sieved, to provide a more homogeneous size distribution of catalyst materials.

Other materials, such as binders, can be added to the precipitated materials. For example, silica can be added as a binder according to methods as are known in the art. Addition of silica as a binder can improve the attrition resistance properties of the formed catalysts.

For example, according to one embodiment a silica binder can be added to the precipitate following filtration and washing of the as-formed precipitate. For instance, a filtered cake of precipitate can be re-slurried in the presence of appropriate amount of SiO₂ sol solution, optionally with the addition of a potassium carbonate (K₂CO₃) solution. As is known in the art, the exact proportion of silica binder added to a FTS catalyst can vary and can, depending upon the relative proportions of materials included in the catalyst, not only improve the attrition resistance of the catalyst, but can also improve catalyst activity. For instance, as described by Hou, et al. (Catal. Lett. (2007) 119:353-360), the increase of the ratio of precipitated SiO₂ (SiO₂(P)) to binder SiO₂ (SiO₂(B)) to about 15:10 can decrease the crystallite size of the catalysts, can improve the surface basicity, can enhance the reduction and carburization of the catalysts, and can increase the activity of the catalyst. However, when the SiO₂(P)/SiO₂(B) ratio is further increased to about 25:0, the catalysts can exhibit a restrained reduction and carburization behavior.

Following initial formation, e.g., co-precipitation, impregnation, deposition, etc. of the precursor materials, the materials can be dried and heat treated. For example, the formed precursor materials can be first dried and then calcined in an oxygen-containing atmosphere to convert the precursors to the metal oxides. For instance, the formed precursors can be dried in an oven, typically at a temperature between about 90° C. and about 110° C. for an appropriate amount of time (e.g., about 1 to about 24 hours). The present disclosure is not limited to any particular drying method, however, and other drying processes are encompassed by the present disclosure. For instance, in one embodiment the precursors can be spray dried.

Heat treatment can take place in air at a temperature higher than that of any drying processes, generally between about 150° C. to about 600° C., or in another embodiment between about 300° C. and about 500° C. Upon heat treatment, metal precursors in the homogeneous precipitated materials can be converted to the metal oxides.

Prior to utilization in an FTS process, a catalyst as described herein can be activated under reducing conditions. The reducing conditions can be provided by heating the materials in a reducing atmosphere of, e.g., hydrogen, carbon monoxide or a combination thereof. For instance, the catalyst can be activated by heating at a temperature of between about 200° C. and about 350° C., more commonly between about 280° C. and about 320° C., in an atmosphere of syngas, optionally at increased pressure.

Disclosed catalysts can be utilized in any FTS process. For instance, disclosed catalysts can be utilized in two-phase FTS processes, in which both the reactants and products are in a gas phase and the solid catalyst forms the second phase, or in three-phase FTS processes, in which products can include liquid hydrocarbon products. Accordingly, FTS processes incorporating the disclosed catalysts can be carried out in fluidized bed reactors, slurry bed reactors, or other suitable reactors, with preferred reactor type generally depending upon the characteristics of the products desired to be formed during the FTS process.

Catalysts can be specifically designed in coordination with the reaction parameters of an FTS process so as to provide the desired products. For instance, the relative proportion of zirconium and iron can be adjusted to alter the activity of the catalysts and as such the proportional hydrocarbon concentrations in the products. Similarly, the catalysts can include one or more additional materials as disclosed previously so as to provide specific catalytic activity to the catalysts. Such variations and corresponding effect on catalytic activity of the disclosed materials as well as effect on proportions obtained of FTS products are well within the ordinary skill level of one in the art, however, and as such are not discussed at length herein.

The presently disclosed subject matter may be better understood by reference to the examples set forth below.

### Example 1

Catalysts were prepared according to the general molar formulation: (100−x)Fe/xZr/5Cu/17Si/4.2K, where x is 5 mol % and the mol % values for Cu, Si, and K are provided based upon 100 mol of Fe (unless otherwise noted, concentration values are provided as molar concentrations).

Initially, ZrO(NO₃)₂ was added to 60 ml of H₂O and heated and stirred until the ZrO(NO₃)₂ was completed dissolved. Subsequently, Fe(NO₃)₃.9H₂O (˜0.6 M) and CuN₂O₆.3H₂O were added to the solution containing Zr. In a second solution, tetraethylorthosilicate (Si(OC₂H₅)₄, TEOS) was added into 40 ml of propanol. Then the solutions were mixed together and heated to 83±3° C. After reaching the targeted temperature, hot, aqueous NH₄OH (˜2.7 M, heated to 83±3° C.) was slowly added to the mixed solution containing the Fe, Cu, Si and Zr precursors under vigorous stirring until the precipitate formed and pH was 8-9. The precipitate was aged in a vessel at room temperature for 17 hours and thoroughly washed with deionized water to remove excess NH₃ and obtain the pH of 7-8 (1.3-1.5 liters of deionized water used). The washed precipitate was dried in an oven for 18-24 hours at 110° C. and was sieved at <90 mm. The resulting catalyst obtained following sizing was incipient impregnated with KHCO₃ solution at the desired ratio. Subsequently, the sample was dried in an oven over 4 hours prior to calcination. The catalyst precursor was calcined in static air at 300° C. for 5 h, and then cooled to room temperature over a 2 hour period in a muffle furnace.

For comparison, other catalysts were prepared using other transition metals in place of the Zr as well as a catalyst from iron alone. Specifically, similar processes were used to form catalysts having an Fe:Me ratio of 95:5 in which the metal, Me, was chromium (Cr), molybdenum (Mo), manganese (Mn), tantalum (Ta), vanadium (V), and tungsten (W).

Following formation, the prepared catalyst was activated in situ under H₂ for 12 hours prior to CO hydrogenation. Feed syngas had an H₂:CO ratio of 2:1. The FTS reaction was carried out in a differential plug-flow reactor at 280° C. and 1.8 atm.

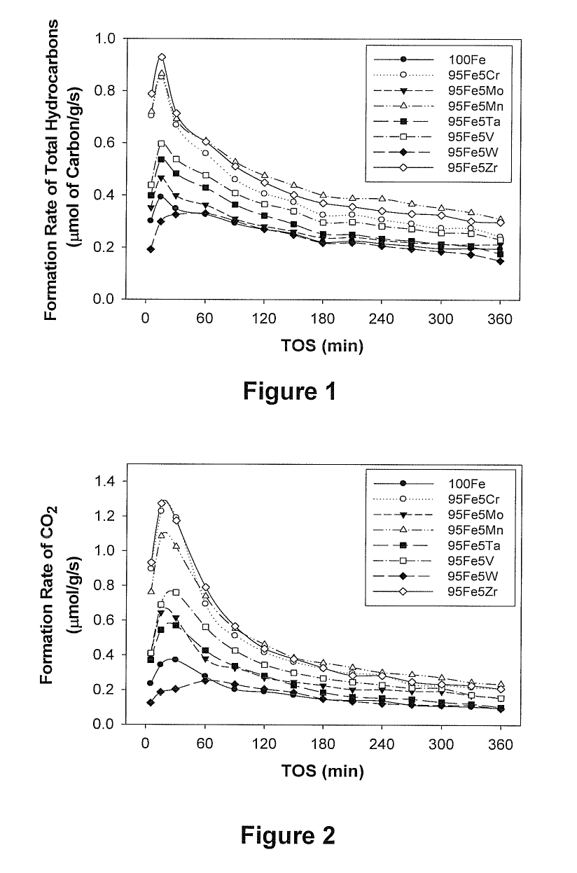

Results showing the activities of the various catalysts thus formed are illustrated in FIGS. 1 and 2. Specifically, FIG. 1 illustrates formation rate of total hydrocarbons vs. time on stream (TOS) and FIG. 2 shows the CO₂ formation rate (WGS) vs. TOS. As can be seen, addition of a transition metal improved catalyst activity for both total hydrocarbon formation and the WGS reaction, with the exception of tungsten. Catalyst activity was most enhanced by the addition of zirconium. The Fe/Zr catalyst did not show any significant induction period behavior for the FTS activity as was observed for the other catalysts.

### Example 2

Catalysts were formed according to precipitation processes as described above for Example 1. Specifically, catalysts were formed according to co-precipitation formation methods as described above and having the following molar formulas:

100Fe/5Cu/17Si

90Fe/10Zr/5Cu/17Si, and

80Fe/20Mn/5Cu/17Si

Following formation, catalysts were impregnated with potassium as described above in Example 1 to include 4.2 mol % potassium by weight of the catalyst.

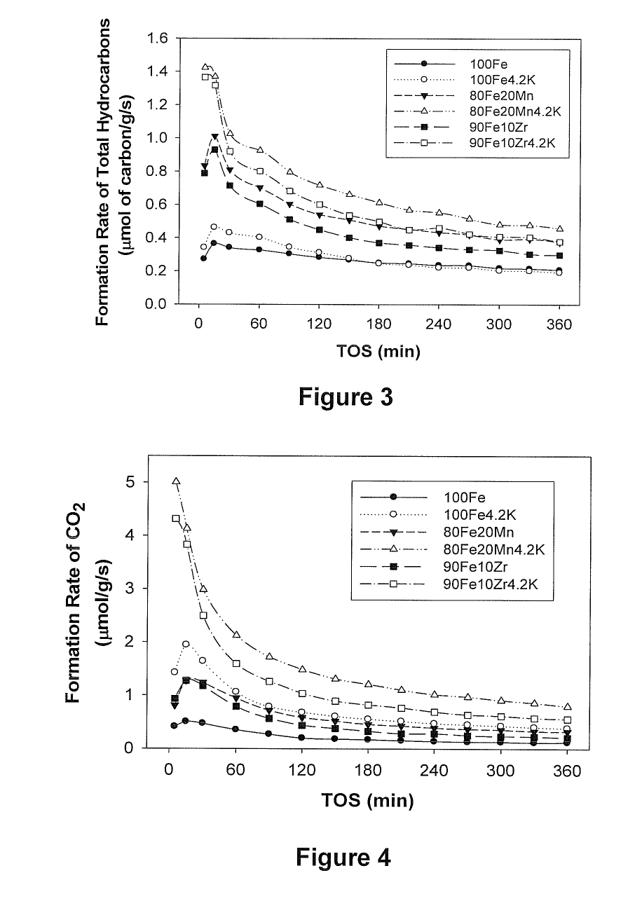

Following formation, the catalysts were activated and utilized in an FTS process as described above in Example 1. Table 1, below, and FIG. 3 illustrate the formation rates and selectivity obtained for total hydrocarbon formation.

TABLE 1

| | max. rate | | | | | | | | |
|---|---|---|---|---|---|---|---|---|---|
| | (mmol | | | | | | | | |
| | C/g/s) | | | | | | | % | |
| | | total | % Hydrocarbon Selectivity | | | | | olefin | a |
| Catalyst | CO₂ | HC | C₁ | C₂ | C₃ | C₄ | C₅-C₈ | (C₂-C₄) | (C₃-C₆) |
| 100Fe/5Cu/17Si | 0.40 | 0.35 | 28.9 | 20.6 | 27.7 | 14.9 | 8.0 | 63 | 0.28 |
| 100Fe/4.2K/5Cu/17Si | 1.94 | 0.46 | 18.0 | 17.4 | 22.9 | 18.8 | 23.0 | 79 | 0.52 |
| 90Fe/10Zr/5Cu/17Si | 1.75 | 1.18 | 32.2 | 18.1 | 23.4 | 14.9 | 10.5 | 52 | 0.4 |
| 90Fe/10Zr/4.2K/5Cu/17Si | 3.83 | 1.36 | 26.5 | 16.9 | 23.1 | 19.4 | 14.1 | 78 | 0.43 |
| 80Fe/20Mn/5Cu/17Si | 1.28 | 1.01 | 34.4 | 17.5 | 23.7 | 16.9 | 7.4 | 65 | 0.36 |
| 80Fe/20Mn/4.2K/5Cu/17Si | 5.00 | 1.42 | 21.2 | 15.3 | 21.5 | 19.7 | 22.3 | 88 | 0.52 |

FIG. 4 illustrates the effect of addition of potassium to the catalysts on WGS CO₂ formation rates and Table 2, below, describes steady-state (SS) formation rates and selectivity for CO₂ and hydrocarbons.

TABLE 2

| | SS rate | | | | | | | | |
|---|---|---|---|---|---|---|---|---|---|
| | (mmol | | | | | | | | |
| | C/g/s) | | | | | | | % | |
| | | total | % Hydrocarbon Selectivity | | | | | olefin | a |
| Catalyst | CO₂ | HC | C₁ | C₂ | C₃ | C₄ | C₅-C₈ | (C₂-C₄) | (C₃-C₆) |
| 100Fe/5Cu/17Si | 0.09 | 0.19 | 26.1 | 29.4 | 26.3 | 13.3 | 4.8 | 74 | 0.29 |
| 100Fe/4.2K/5Cu/17Si | 0.43 | 0.20 | 21.2 | 28.2 | 23.8 | 10.4 | 16.4 | 79 | 0.46 |
| 90Fe/10Zr/5Cu/17Si | 0.32 | 0.33 | 31.4 | 26.8 | 23.5 | 12 | 6.4 | 74 | 0.34 |
| 90Fe/10Zr/4.2K/5Cu/17Si | 0.61 | 0.41 | 27.5 | 25.8 | 19.9 | 18.8 | 7 | 83 | 0.42 |
| 80Fe/20Mn/5Cu/17Si | 0.35 | 0.39 | 33.7 | 26.5 | 22.1 | 15.8 | 2.0 | 83 | 0.27 |
| 80Fe/20Mn/4.2K/5Cu/17Si | 0.91 | 0.48 | 23.9 | 23.9 | 21.9 | 19.9 | 10.5 | 91 | 0.41 |

As can be seen the addition of potassium to the disclosed catalysts can alter catalyst activity during FTS processes.

### Example 3

Fe Catalysts were formed according to precipitation processes as described above for Example 1. Specifically, catalysts were formed according to co-precipitation formation methods as described above and having the following molar formulas:

100Fe

100Fe/4.2K

80Fe/20Mn

80Fe/20Mn/4.2K

90Fe/10Zr

90Fe/10Zr/4.2K

Following formation, the catalysts were activated and utilized in an FTS process as described above in Example 1. Table 3, below illustrates the catalyst activities and selectivities obtained for total hydrocarbon formation.

TABLE 3

| | Max rate^{b} | | SS rate^{b,c} | | | | | | | | |
|---|---|---|---|---|---|---|---|---|---|---|---|
| | (μmol of | | (μmol of | | % Hydrocarbon | | | | | % | |
| | C/g/s) | | C/g/s) | | Selectivity | | | | | olefin^{b,c} | |
| | | Total | | Total | at SS^{b,c,e} | | | | | (C₂-C₄ | a^{b,c} |
| Catalyst^{a} | CO₂ | HC | CO₂ | HC | C₁ | C₂ | C₃ | C₄ | C₅-C₈ | fraction) | (C₃-C₆) |
| 100Fe | 0.50 | 0.78 | 0.13 | 0.43 | 27 | 29 | 23 | 16 | 5 | 74 | 0.29 |
| 100Fe/4.2K | 2.01 | 1.17 | 0.48 | 0.44 | 19 | 25 | 22 | 16 | 17 | 95 | 0.45 |
| 80Fe/20Mn | 1.27 | 1.78 | 0.35 | 0.77 | 34 | 26 | 22 | 16 | 2 | 83 | 0.27 |
| 80Fe/20Mn/4.2K | 3.98 | 3.14 | 0.91 | 1.07 | 24 | 24 | 22 | 20 | 10 | 91 | 0.41 |
| 90Fe/10Zr | 1.75 | 2.24 | 0.32 | 0.58 | 31 | 27 | 24 | 12 | 6 | 82 | 0.34 |
| 90Fe/10Zr/4.2K | 4.31 | 2.91 | 0.61 | 0.79 | 26 | 26 | 20 | 21 | 5 | 85 | 0.42 |

^{a}all catalysts also contained 5Cu and 17Si
^{b}Max error = ±5%
^{c}at steady-state rate (% h TOS)
^{e}based on atomic carbon

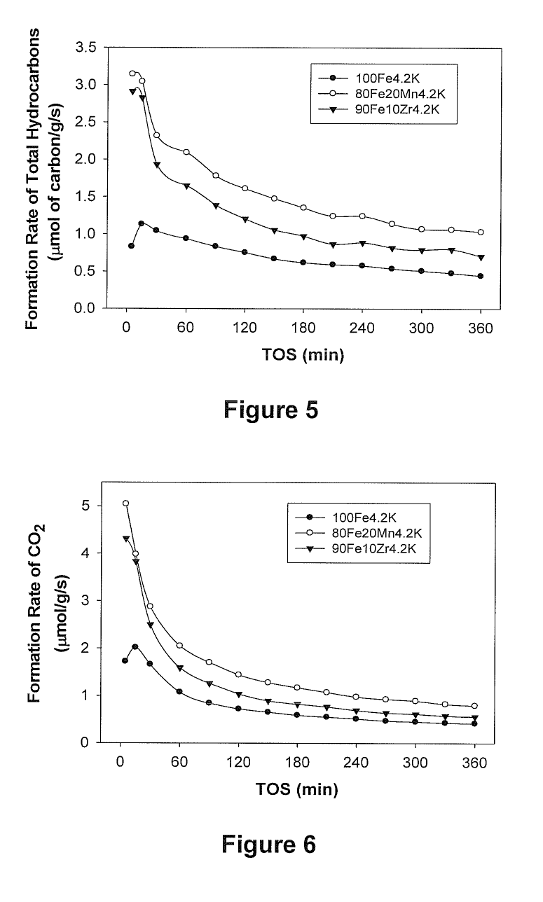

FIG. 5 illustrates the impact of the potassium addition to the catalysts with regard to the formation rate of total hydrocarbons, and FIG. 6 illustrates the impact of the potassium addition with regard to the formation rate of carbon dioxide.

It will be appreciated that the foregoing examples, given for purposes of illustration, are not to be construed as limiting the scope of this disclosure. Although only a few exemplary embodiments of the disclosed subject matter have been described in detail above, those skilled in the art will readily appreciate that many modifications are possible in the exemplary embodiments without materially departing from the novel teachings and advantages of this disclosure. Accordingly, all such modifications are intended to be included within the scope of this disclosure. Further, it is recognized that many embodiments may be conceived that do not achieve all of the advantages of some embodiments, yet the absence of a particular advantage shall not be construed to necessarily mean that such an embodiment is outside the scope of the present disclosure.

What is claimed is:

1. A Fischer-Tropsch synthesis catalyst comprising zirconium inserted throughout an iron oxide bulk catalyst, the catalyst including zirconium in an amount between about 3 mol % and about 20 mol % of the total amount of iron and zirconium in the catalyst, wherein the catalyst is a titanium-free catalyst.

2. The Fischer-Tropsch synthesis catalyst of claim 1, the catalyst further comprising an additional metal or metalloid.

3. The Fischer-Tropsch synthesis catalyst of claim 2, wherein the additional metal or metalloid is selected from the group consisting of silicon, copper, manganese, chromium, cobalt, nickel, vanadium, tantalum, potassium, sodium, cesium, and molybdenum.

4. The Fischer-Tropsch synthesis catalyst of claim 2, the catalyst further comprising potassium.

5. The Fischer-Tropsch synthesis catalyst of claim 1, the catalyst defining a surface area of about 350 square meters per gram.

6. The Fischer-Tropsch synthesis catalyst of claim 1, wherein the catalyst is a particulate.

7. The Fischer-Tropsch synthesis catalyst of claim 6, wherein the catalyst comprises particles less than about 150 μm in size.

8. The Fischer-Tropsch synthesis catalyst of claim 6, wherein the catalyst comprises particles between about 50 μm and about 100 μm in size.

9. The Fischer-Tropsch synthesis catalyst of claim 1, wherein the catalyst is a bi-metallic catalyst.

10. The Fischer-Tropsch synthesis catalyst of claim 1, the catalyst further comprising a binder.

11. The Fischer-Tropsch synthesis catalyst of claim 10, wherein the binder is a silica binder.

12. A method for forming a Fischer-Tropsch synthesis catalyst comprising
forming a titanium-free mixture including a zirconium containing precursor and an iron containing precursor in solution, the mixture including between about 3 mol % and about 20 mol % zirconium as compared to the total amount of iron and zirconium in the mixture;
adding a precipitation initiator to the mixture; and
co-precipitating the iron and the zirconium to form a particulate material including zirconium inserted throughout the bulk iron precipitate.

13. The method according to claim 12, wherein the zirconium containing precursor and the iron containing precursor are salts.

14. The method according to claim 12, wherein the zirconium containing precursor and the iron containing precursor are independently selected from the group consisting of metal nitrates, oxalates, sulphates, chlorides, alkoxides, acetates, benzoates, and maleates.

15. The method according to claim 12, wherein the iron and zirconium are co-precipitated at a temperature of between about 50° C. and about 100° C.

16. The method according to claim 12, the mixture further comprising an additional compound, the method further comprising co-precipitating the additional compound with the zirconium and the iron.

17. The method according to claim 16, wherein the additional compound is copper or silicon.

18. The method according to claim 12, further comprising depositing or impregnating the particulate material with an additional material.

19. The method according to claim 18, wherein the additional material comprises potassium.

20. The method according to claim 18, wherein the additional material is a silica binder.

21. The method according to claim 12, further comprising converting the iron to iron oxide and the zirconium to zirconium oxide.

22. The method according to claim 21, further comprising activating the particulate material under reducing conditions.

23. A method for converting reactants comprising
contacting a gas stream comprising carbon monoxide and hydrogen gas with a titanium-free catalyst, the titanium-free catalyst including zirconium inserted throughout an iron oxide bulk catalyst, the titanium-free catalyst including the zirconium in an amount of between about 3 mol % and about 20 mol % of the total amount of iron and zirconium in the catalyst; and
converting the carbon monoxide and the hydrogen gas into hydrocarbons according to a Fischer-Tropsch synthesis process.

24. The method according to claim 23, wherein the hydrocarbons are gaseous.

25. The method according to claim 23, wherein the hydrocarbons comprise liquid hydrocarbons.
